SEQUENCE LISTING (8 sequences)

SEQ ID NO: 1 - DNA, 14 bp, synthetic construct
  source 1..14: mol_type = other DNA
tcgtcggcagcgtc

SEQ ID NO: 2 - DNA, 15 bp, synthetic construct
  source 1..15: mol_type = other DNA
gtctcgtgggctcgg

SEQ ID NO: 3 - DNA, 19 bp, synthetic construct
  source 1..19: mol_type = other DNA
agatgtgtataagagacag

SEQ ID NO: 4 - DNA, 33 bp, synthetic construct
  source 1..33: mol_type = other DNA
tcgtcggcagcgtcagatgtgtataagagacag

SEQ ID NO: 5 - DNA, 34 bp, synthetic construct
  source 1..34: mol_type = other DNA
gtctcgtgggctcggagatgtgtataagagacag

SEQ ID NO: 6 - DNA, 17 bp, synthetic construct
  source 1..17: mol_type = other DNA
cgacgctcttccgatct

SEQ ID NO: 7 - DNA, 18 bp, synthetic construct
  source 1..18: mol_type = other DNA
cgtgtgctcttccgatct

SEQ ID NO: 8 - DNA, 19 bp, synthetic construct
  source 1..19: mol_type = other DNA
ctgtctcttatacacatct

(19) United States
(12) Patent Application Publication
(10) Pub. No.: US 2024/0327904 A1
(43) Pub. Date: Oct. 3, 2024

(54) CELL FIXATIVE AGENTS FOR SINGLE CELL SEQUENCING

(71) Applicant: Bio-Rad Laboratories, Inc., Hercules, CA (US)

(72) Inventors: Man CHENG, Danville, CA (US); Qiang ZHANG, Pleasanton, CA (US); Linda HINH, Danville, CA (US)

(21) Appl. No.: 18/619,911

(22) Filed: Mar. 28, 2024

Related U.S. Application Data
(60) Provisional application No. 63/455,903, filed on Mar. 30, 2023.

(51) Int. Cl. C12Q 1/6851; C12Q 1/6806

(52) U.S. Cl. CPC C12Q 1/6851; C12Q 1/6806

(57) ABSTRACT

Compositions and methods for fixing cells or nuclei or extracellular vesicles with dithiobismaleimidoethane (DTME) and subsequent reverse transcription in the cells or nuclei or extracellular vesicles are provided.

### CROSS-REFERENCE TO RELATED PATENT APPLICATIONS

The present patent application claims benefit of priority to U.S. Provisional Patent Application No. 63/455,903, filed Mar. 30, 2023, which is incorporated by reference for all purposes.

### SEQUENCE LISTING

The instant application contains a Sequence Listing which has been submitted electronically in XML file format and is hereby incorporated by reference in its entirety. Said XML copy, created on Apr. 25, 2024, is named 094868-1427713_121310US_SL.xml and is 8,095 bytes in size.

### BACKGROUND OF THE INVENTION

Cell fixation chemically alters components in a cell to reduce or prevent cell lysis or degradation, allowing one to molecularly examine target components within the cell. Fixation can therefore allow for various in situ methods of analysis that might not otherwise be possible. Cell fixation can result in cross-linking and other chemical alterations and thus in some cases the chemical alterations can interfere with a method of analysis.

### BRIEF SUMMARY OF THE INVENTION

In some embodiments, a method of in situ reverse transcription is provided. In some embodiments, the method comprises, contacting a single cell or a plurality of cells or nuclei or extracellular vesicles with dithiobismaleimidoethane (DTME) in an amount sufficient to fix the cells; permeabilizing the cells or nuclei or extracellular vesicles; and introducing a reverse transcriptase into the cells or nuclei or extracellular vesicles to form first strand cDNAs from the RNAs in the cells or nuclei or extracellular vesicles, thereby conducting in situ reverse transcription. In some embodiments, the RNAs are mRNAs. In some embodiments, the plurality of cells is in a plant tissue or animal tissue.

In some embodiments, the cells are individual cells and the individual cells are within separate partitions. In some embodiments, the partitions are droplets, tubes, gel beads or microwells. In some embodiments, the method comprises linking partition-specific barcode oligonucleotides to the cDNAs or fragments or complements thereof.

In some embodiments, the method further comprises amplifying the cDNAs or fragments thereof with a polymerase. In some embodiments, the amplifying comprises polymerase chain reaction (PCR). In some embodiments, the PCR is monitored in real-time.

In some embodiments, the RNAs and first strand cDNAs form RNA/DNA hybrids and the method further comprising contacting the RNA/DNA hybrids with contacting a transposase that introduces heterologous end adaptor oligonucleotides into the RNA/DNA hybrids to form DNA fragments, and optionally RNA fragments, comprising the heterologous end adaptor oligonucleotides.

In some embodiments, the method further comprises the forming second strand cDNAs from the first strand cDNAs wherein the first strand cDNAs and second strand cDNAs form double-stranded DNA and the method further comprising contacting the double-stranded DNA with contacting a transposase that introduces heterologous end adaptor oligonucleotides into the double-stranded DNA to form DNA fragments comprising the heterologous end adaptor oligonucleotides.

In some embodiments, the cells are mammalian cells.

In some embodiments, the method comprises storing the cells (e.g., for at least 12 or 24 hours or 1, 2, 3, 4, 5, 10, 20, or 30 days, optionally no more than double or triple those periods of time) after the fixing and before the permeabilizing or the introducing.

In some embodiments, the RNAs and first strand cDNAs form RNA/DNA hybrids and the method further comprises:
introducing into the cells and nuclei of the cells transposases, wherein the transposases:
(i) introduce heterologous end adaptor oligonucleotides into genomic DNA in the nuclei to form gDNA fragments comprising the heterologous end adaptor oligonucleotides; and
(ii) introduces heterologous end adaptor oligonucleotides into the RNA/DNA hybrids to form cDNA fragments comprising the heterologous end adaptor oligonucleotides; introducing the cells into partitions;
in the partitions, attaching partition-specific barcode oligonucleotides to the gDNA fragments comprising the heterologous end adaptor oligonucleotides and the cDNA fragments comprising the heterologous end adaptor oligonucleotides.

In some embodiments, a plurality of fixed cells is provided, wherein the cells were fixed with dithiobismaleimidoethane (DTME) and the fixed cells comprise a heterologous reverse transcriptase capable of generating cDNAs from mRNAs in the fixed cells. In some embodiments, the cells are mammalian cells.

In some embodiments, individual cells of the plurality are contained in separate partitions. In some embodiments, the partitions are droplets or microwells.

### Definitions

Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well-known and commonly employed in the art.

The term “amplification reaction” refers to any in vitro means for multiplying the copies of a target sequence of nucleic acid in a linear or exponential manner. Such methods include but are not limited to polymerase chain reaction (PCR); DNA ligase chain reaction (see U.S. Pat. Nos. 4,683,195 and 4,683,202; PCR Protocols: A Guide to Methods and Applications (Innis et al., eds, 1990)) (LCR); QBeta RNA replicase and RNA transcription-based amplification reactions (e.g., amplification that involves T7, T3, or SP6 primed RNA polymerization), such as the transcription amplification system (TAS), nucleic acid sequence based amplification (NASBA), and self-sustained sequence replication (3SR); isothermal amplification reactions (e.g., single-primer isothermal amplification (SPIA)); as well as others known to those of skill in the art.

“Amplifying” refers to a step of submitting a solution to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing or linear amplification. In an exemplary embodiment, amplifying refers to PCR amplification using a first and a second amplification primer.

The term “amplification reaction mixture” refers to an aqueous solution comprising the various reagents used to amplify a target nucleic acid. These include enzymes, aqueous buffers, salts, amplification primers, target nucleic acid, and nucleoside triphosphates. Amplification reaction mixtures may also further include stabilizers and other additives to optimize efficiency and specificity. Depending upon the context, the mixture can be either a complete or incomplete amplification reaction mixture.

“Polymerase chain reaction” or “PCR” refers to a method whereby a specific segment or subsequence of a target double-stranded DNA, is amplified in a geometric progression. PCR is well known to those of skill in the art; see, e.g., U.S. Pat. Nos. 4,683,195 and 4,683,202; and PCR Protocols: A Guide to Methods and Applications, Innis et al., eds, 1990. Exemplary PCR reaction conditions typically comprise either two or three step cycles. Two step cycles have a denaturation step followed by a hybridization/elongation step. Three step cycles comprise a denaturation step followed by a hybridization step followed by a separate elongation step.

A “primer” refers to a polynucleotide sequence that hybridizes to a sequence on a target nucleic acid and serves as a point of initiation of nucleic acid synthesis. Primers can be of a variety of lengths and are often less than 50 nucleotides in length, for example 12-30 nucleotides, in length. The length and sequences of primers for use in PCR can be designed based on principles known to those of skill in the art, see, e.g., Innis et al., supra. Primers can be DNA, RNA, or a chimera of DNA and RNA portions. In some cases, primers can include one or more modified or non-natural nucleotide bases. In some cases, primers are labeled.

“Primer extension” refers to any method in which a primer is extended in a template-specific manner. Examples of primer extension include, for example, methods in which a primer hybridizes to a template nucleic acid and a polymerase extends the primer in a template-specific manner. In some embodiments, the template is DNA and the polymerase is a DNA polymerase. In some embodiments, the template is RNA and the polymerase is a reverse-transcriptase. Primer extension can also include, for example, template switching (see, e.g., Zhu Y Y, Machleder E M, et al. (2001) Biotechniques, 30(4):892-897; Ramskold D, Luo S, et al. (2012) Nat Biotechnol, 30(8):777-78, and nick polymerization (also referred to as nick translation), the latter involving nicking one strand of a nucleic acid duplex and using the nicked strand as a primer that is extended using the other strand as a template (see, e.g., Leonard G. Davis Ph.D., et al, in Basic Methods in Molecular Biology, 1986).

A nucleic acid, or a portion thereof, “hybridizes” or “anneals” to another nucleic acid under conditions such that non-specific hybridization is minimal at a defined temperature in a physiological buffer (e.g., pH 6-9, 25-150 mM chloride salt or in a PCR reaction mixture). In some cases, a nucleic acid, or portion thereof, hybridizes to a conserved sequence shared among a group of target nucleic acids. In some cases, a primer, or portion thereof, can hybridize to a primer binding site if there are at least about 6, 8, 10, 12, 14, 16, or 18 contiguous complementary nucleotides, including “universal” nucleotides that are complementary to more than one nucleotide partner. Alternatively, a primer, or portion thereof, can hybridize to a primer binding site if there are fewer than 1 or 2 complementarity mismatches over at least about 12, 14, 16, or 18 contiguous complementary nucleotides. In some embodiments, the defined temperature at which specific hybridization occurs is room temperature. In some embodiments, the defined temperature at which specific hybridization occurs is higher than room temperature. In some embodiments, the defined temperature at which specific hybridization occurs is at least about 37, 40, 42, 45, 50, 55, 60, 65, 70, 75, or 80° C. In some embodiments, the defined temperature at which specific hybridization occurs is 37, 40, 42, 45, 50, 55, 60, 65, 70, 75, or 80° C.

A “template” refers to a polynucleotide sequence that comprises the polynucleotide to be amplified, flanked by or a pair of primer hybridization sites. Thus, a “target template” comprises the target polynucleotide sequence adjacent to at least one hybridization site for a primer. In some cases, a “target template” comprises the target polynucleotide sequence flanked by a hybridization site for a “forward” primer and a “reverse” primer.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications including but not limited to capping with a fluorophore (e.g., quantum dot) or another moiety.

A “polymerase” refers to an enzyme that performs template-directed synthesis of polynucleotides, e.g., DNA and/or RNA. The term encompasses both the full length polypeptide and a domain that has polymerase activity. DNA polymerases are well-known to those skilled in the art, including but not limited to DNA polymerases isolated or derived from Pyrococcus furiosus, Thermococcus litoralis, and Thermotoga maritime, or modified versions thereof. Additional examples of commercially available polymerase enzymes include, but are not limited to: Klenow fragment (New England Biolabs® Inc.), Taq DNA polymerase (QIAGEN), 9° N™ DNA polymerase (New England Biolabs® Inc.), Deep Vent™ DNA polymerase (New England Biolabs® Inc.), Manta DNA polymerase (Enzymatics®), Bst DNA polymerase (New England Biolabs® Inc.), and phi29 DNA polymerase (New England Biolabs® Inc.).

Polymerases include both DNA-dependent polymerases and RNA-dependent polymerases such as reverse transcriptase. At least five families of DNA-dependent DNA polymerases are known, although most fall into families A, B and C. Other types of DNA polymerases include phage polymerases. Similarly, RNA polymerases typically include eukaryotic RNA polymerases I, II, and III, and bacterial RNA polymerases as well as phage and viral polymerases. RNA polymerases can be DNA-dependent and RNA-dependent.

As used herein, the term “partitioning” or “partitioned” refers to separating a sample into a plurality of portions, or “partitions.” Partitions are generally physical, such that a sample in one partition does not, or does not substantially, mix with a sample in an adjacent partition. Partitions can be solid or fluid. In some embodiments, a partition is a solid partition, e.g., a microchannel. In some embodiments, a partition is a fluid partition, e.g., a droplet. In some embodiments, a fluid partition (e.g., a droplet) is a mixture of immiscible fluids (e.g., water and oil). In some embodiments, a fluid partition (e.g., a droplet) is an aqueous droplet that is surrounded by an immiscible carrier fluid (e.g., oil).

As used herein a “barcode” is a short nucleotide sequence (e.g., at least about 4, 6, 8, 10, 12, 14, 16, 18, 20 or more nucleotides long) that identifies a molecule to which it is conjugated. Barcodes can be used, e.g., to identify molecules in a partition. Such a partition-specific barcode should be unique for that partition as compared to barcodes present in other partitions. For example, partitions containing target RNA from single-cells can be subject to reverse transcription conditions using primers that contain a different partition-specific barcode sequence in each partition, thus incorporating a copy of a unique “cellular barcode” or “partition-specific barcode” into the reverse transcribed nucleic acids of each partition. Thus, nucleic acid from each cell can be distinguished from nucleic acid of other cells due to the unique “cellular barcode.” In some cases, the cellular barcode is provided by a “bead barcode” that is present on oligonucleotides conjugated to a bead, wherein the bead barcode is shared by (e.g., identical or substantially identical amongst) all, or substantially all, of the oligonucleotides conjugated to that bead but is different from most or substantially all oligonucleotides conjugated to other beads. Thus, cellular and bead barcodes can be present in a partition, attached to a bead, or bound to cellular nucleic acid as multiple copies of the same barcode sequence. Cellular or bead barcodes of the same sequence can be identified as deriving from the same cell, partition, or bead. Such partition-specific, cellular, or bead barcodes can be generated using a variety of methods, which methods result in the barcode conjugated to or incorporated into a solid or hydrogel support (e.g., a solid bead or particle or hydrogel bead or particle). In some cases, the partition-specific, cellular, or bead barcode is generated using a split and mix (also referred to as split and pool) synthetic scheme as described herein. A partition-specific barcode can be a cellular barcode and/or a bead barcode (for example when associated with a cell or partition or both). Similarly, a cellular barcode can be a partition specific barcode (when provided in a partition) and/or a bead barcode (when delivered by a bead). Additionally, a bead barcode can be a cellular barcode and/or a partition-specific barcode.

In other cases, barcodes uniquely identify the molecule to which it is conjugated and are referred to as a unique molecular identifier (UMI). The number of nucleotides of the UMI, which can be continuous, or discontinuous, will depend on the number of UMI sequences required. In some embodiments, the number of UMIs available are many times (e.g., 2×, 10×, 100×, etc) higher than possible conjugation partners, thereby reducing the chance of rare duplicates being linked to different molecules. In some embodiments, pools of different UMIs are present in a partition and the composition of the pool acts as an identifiers for the partition, with some UMIs being in common with some other partitions but the total pool of UMIs being unique or substantially unique between partitions. UMI sequences can be generated for example as random sequences of a set length, and in some embodiments is identified by a flanking known sequence. In some cases, UMI and partition-specific barcodes as well as other types of barcodes (e.g., sample barcodes or other barcodes) are ultimately linked to target nucleic acid fragments.

The length of the barcode sequence determines how many unique samples can be differentiated. For example, a 1 nucleotide barcode can differentiate 4, or fewer, different samples or molecules; a 4-nucleotide barcode can differentiate 4⁴ or 256 samples or less; a 6 nucleotide barcode can differentiate 4096 different samples or less; and an 8 nucleotide barcode can index 65,536 different samples or less. Additionally, barcodes can be attached to both strands either through barcoded primers for both first and second strand synthesis, through ligation, or in a tagmentation reaction.

Barcodes are typically synthesized and/or polymerized (e.g., amplified) using processes that are inherently inexact. Thus, barcodes that are meant to be uniform (e.g., a cellular, particle, or partition-specific barcode shared amongst all barcoded nucleic acid of a single partition, cell, or bead) can contain various N−1 deletions or other mutations from the canonical barcode sequence. Thus, barcodes that are referred to as “identical” or “substantially identical” copies refer to barcodes that differ due to one or more errors in, e.g., synthesis, polymerization, or purification errors, and thus contain various N−1 deletions or other mutations from the canonical barcode sequence. Moreover, the random conjugation of barcode nucleotides during synthesis using e.g., a split and pool approach and/or an equal mixture of nucleotide precursor molecules as described herein, can lead to low probability events in which a barcode is not absolutely unique (e.g., different from all other barcodes of a population or different from barcodes of a different partition, cell, or bead). However, such minor variations from theoretically ideal barcodes do not interfere with the high-throughput sequencing analysis methods, compositions, and kits described herein. Therefore, as used herein, the term “unique” in the context of a particle, cellular, partition-specific, or molecular barcode encompasses various inadvertent N−1 deletions and mutations from the ideal barcode sequence. In some cases, issues due to the inexact nature of barcode synthesis, polymerization, and/or amplification, are overcome by oversampling of possible barcode sequences as compared to the number of barcode sequences to be distinguished (e.g., at least about 2-, 5-, 10-fold or more possible barcode sequences). For example, 10,000 cells can be analyzed using a cellular barcode having 9 barcode nucleotides, representing 262,144 possible barcode sequences. The use of barcode technology is well known in the art, see for example Katsuyuki Shiroguchi, et al. Proc Natl Acad Sci USA., 2012 Jan. 24; 109(4):1347-52; and Smith, A M et al., Nucleic Acids Research Can 11, (2010). Further methods and compositions for using barcode technology include those described in U.S. 2016/0060621.

A “transposase” or “tagmentase” means an enzyme that is capable of forming a functional complex with a transposon end-containing composition and catalyzing insertion or transposition of the transposon end-containing composition into the double-stranded target DNA with which it is incubated in an in vitro transposition reaction.

The term “transposon end” means a double-stranded DNA that exhibits the nucleotide sequences (the “transposon end sequences”) that are necessary to form the complex with the transposase that is functional in an in vitro transposition reaction. A transposon end forms a “complex” or a “synaptic complex” or a “transposome complex” or a “transposome composition” with a transposase or integrase that recognizes and binds to the transposon end, and which complex is capable of inserting or transposing the transposon end into target DNA with which it is incubated in an in vitro transposition reaction. A transposon end exhibits two complementary sequences consisting of a “transferred transposon end sequence” or “transferred strand” and a “non-transferred transposon end sequence,” or “non transferred strand” For example, one transposon end that forms a complex with a hyperactive Tn5 transposase (e.g., EZ-Tn5™ Transposase, EPICENTRE Biotechnologies, Madison, Wis., USA) that is active in an in vitro transposition reaction comprises a transferred strand that exhibits a “transferred transposon end sequence” as follows:

(SEQ ID NO: 3)
5′AGATGTGTATAAGAGACAG 3′,

and a non-transferred strand that exhibits a “non-transferred transposon end sequence” as follows:

(SEQ ID NO: 8)
5′ CTGTCTCTTATACACATCT 3′.

The 3′-end of a transferred strand is joined or transferred to target DNA in an in vitro transposition reaction. The non-transferred strand, which exhibits a transposon end sequence that is complementary to the transferred transposon end sequence, is not joined or transferred to the target DNA in an in vitro transposition reaction.

The term “solid support” refers to the surface of a bead, microtiter well or other surface that is useful for attaching a nucleic acid, such as an oligonucleotide or polynucleotide. The surface of the solid support can be treated to facilitate attachment of a nucleic acid, such as a single stranded nucleic acid.

The term “bead” refers to any solid support that can be in a partition, e.g., a small particle or other solid support. In some embodiments, the beads comprise an alginate matrix, i.e., calcium alginate. In some embodiments, the beads comprise polyacrylamide. For example, in some embodiments, the beads incorporate barcode oligonucleotides into the gel matrix through an acrydite chemical modification attached to each oligonucleotide. Exemplary beads can include hydrogel beads. In some cases, the hydrogel is in sol form. In some cases, the hydrogel is in gel form. An exemplary hydrogel is an agarose hydrogel. Other hydrogels include, but are not limited to, those described in, e.g., U.S. Pat. Nos. 4,438,258; 6,534,083; 8,008,476; 8,329,763; U.S. Patent Appl. Nos. 2002/0,009,591; 2013/0,022,569; 2013/0,034,592; and International Patent Publication Nos. WO/1997/030092; and WO/2001/049240.

It will be understood that any range of numerical values disclosed herein can include the endpoints of the range, and any values or subranges in between the endpoints. For example, the range 1 to 10 includes the endpoints 1 and 10, and any value between 1 and 10. The values typically include one significant digit.

The term “sample” refers to a biological composition, such as a cell, comprising a target nucleic acid.

The term “about” refers to the usual error range for the respective value that is known by a person of ordinary skill in the art for this technical field, for example, a range of ±10%, ±5%, or ±1% can encompass the recited value, even if the recited value is not modified by the term “about.”

All ranges described herein can include the end point values of the range, and any sub-range of values included between the endpoints of the range, where the values include the first significant digit. For example, a range of 1 to 10 includes a range from 2 to 9, 3 to 8, 4 to 7, 5 to 6, 1 to 5, 2 to 5, 2 to 10, 3 to 10, and so on.

As used herein, a polypeptide (e.g., a reverse transcriptase) or a nucleic acid is “heterologous” to a cell or organism if the polypeptide or nucleic acid originates from a foreign species compared to the cell or organism, or, if from the same species, is modified from its original form.

### BRIEF DESCRIPTION OF THE DRAWINGS

FIG. 1a-c show real-time quantitative PCR results of amplicon CNOT4 (a), PUM1 (b), and UBC (c) by using cDNA samples derived from the fixed-cell in situ reverse transcription reaction (Test Sample) and the commercial iScript Advanced cDNA Synthesis Kit (Control Sample), respectively. Reverse transcription of Test and Control samples was conducted in the presence and absence of reverse transcriptase supplement to make sure the amplification is cDNA specific. The amount of cDNA input was normalized per cell-equivalent between Test and Control. The qPCR assays were performed in duplicate with four 10-fold serial diluted inputs of sample to establish a titration curve for amplification efficiency determination. The level of threshold cutoff was determined by the algorithm and fixed among all samples for the Cq value determination and comparison. One representation result of at least three experimental repeats were shown.

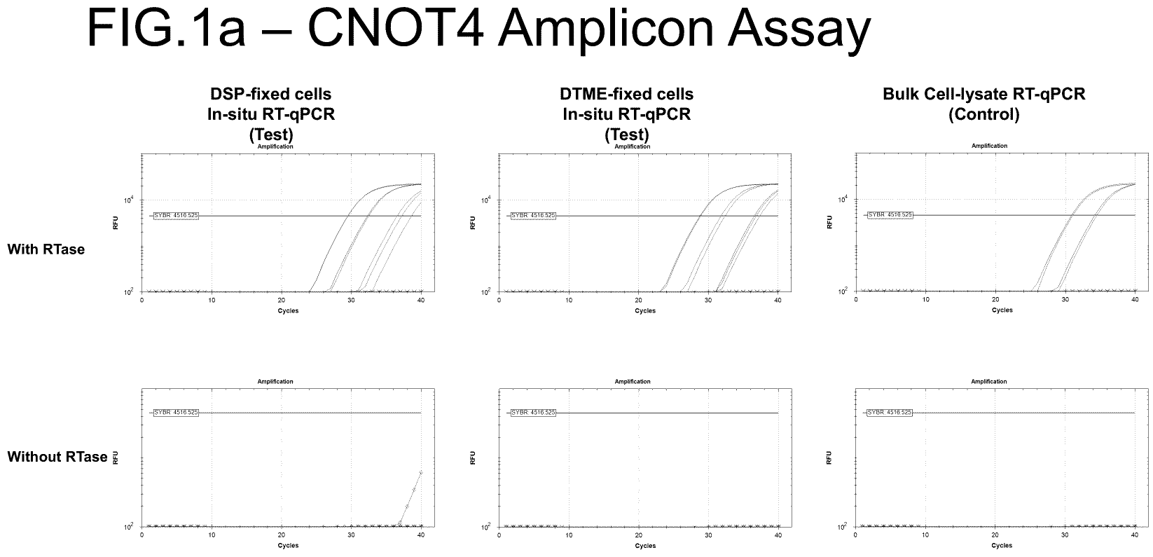

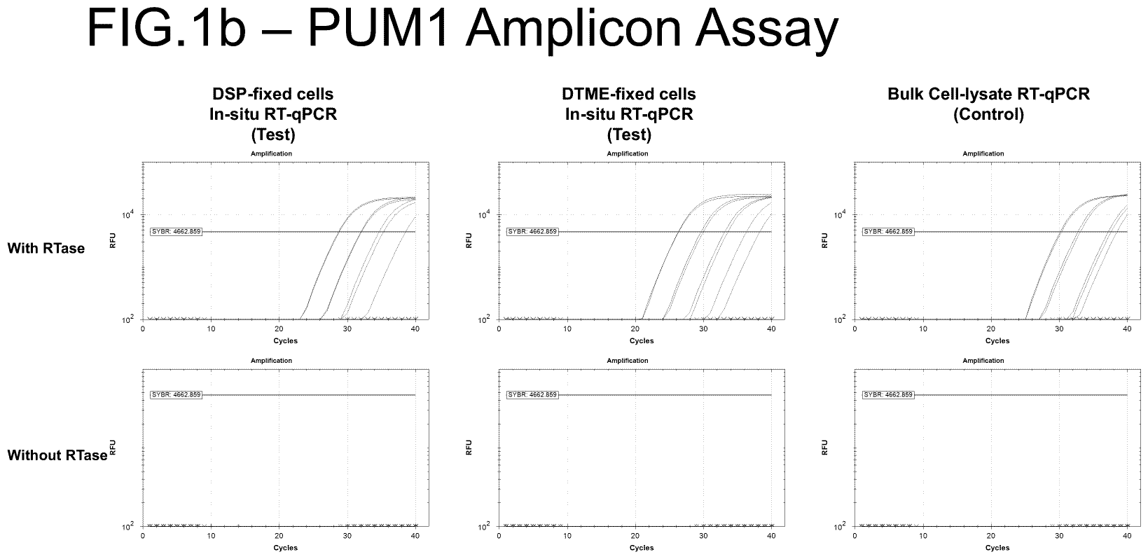

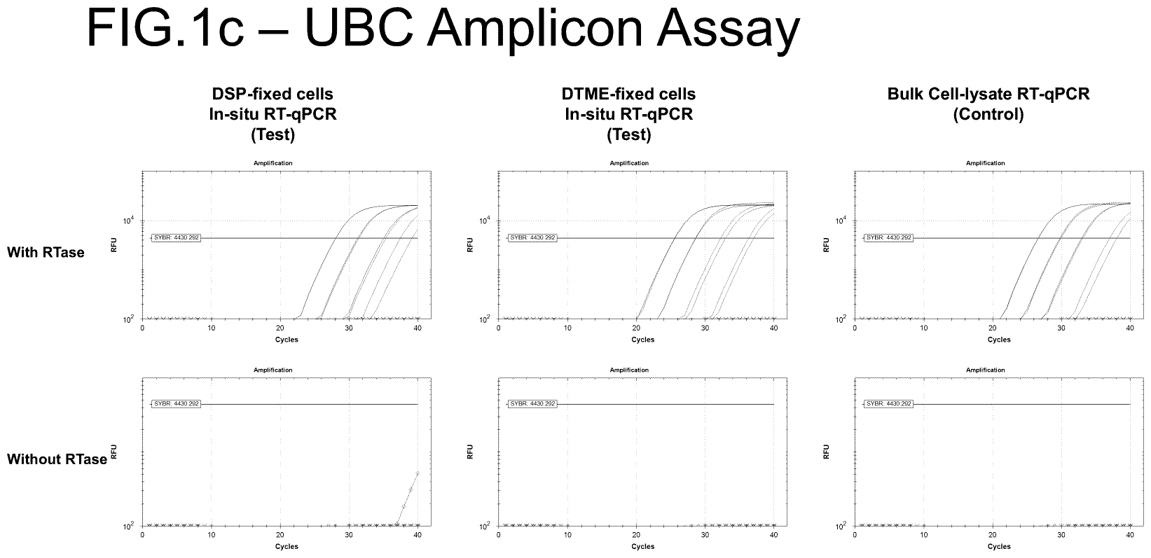

### DETAILED DESCRIPTION OF THE INVENTION

The inventors have found that one can fix cells or nuclei or extracellular vesicles with dithiobismaleimidoethane (DTME). Fixation of cells with DTME allows for greatly improved in situ reverse transcription (RT) in the cells than RT in cells fixed with other tested fixative agents. While dithiobis(succinimidyl propionate) (DSP), or Lomant's Reagent has been previously described to stabilize cell samples for single-cell transcriptomic applications (see, e.g., Attar et al., Scientific Report| (2018) 8:21511, it has been discovered that DTME is superior for various in situ reverse transcription applications including but not limited to for single cell transcriptional applications.

Dithiobismaleimidoethane (DTME), which reacts with sulfhydryl groups, e.g., in proteins and other biomolecules, has been described before, e.g., in the context of fixation for use in mass spectrometry. See, e.g., Smith et al., PLoS ONE 6(1): e16206. Fixation of cells or nuclei or vesicles with DTME can be achieved by incubating the cells or nuclei or vesicles with DTME for a sufficient time to fix the cells. In some embodiments, the cells, nuclei or vesicles are incubated with, e.g., 0.1-10 mM or, e.g., 0.1 to 0.8 mg/ml DTME, and in some embodiments the incubation is between 5-120, e.g., 20-40, e.g., 30 minutes. Fluorescent cell imaging and/or flow cytometry can be conducted to confirm the effectiveness of DTME fixation if desired in a concentration dependent manner.

Any type of cells can be used according to the methods and compositions described herein. In some embodiments, the cells are mammalian, for example human cells. In some embodiments, the cells are from a biological sample. Biological samples can be obtained from any biological organism, e.g., an animal, plant, fungus, pathogen (e.g., bacteria or virus), or any other organism. In some embodiments, the biological sample is from an animal, e.g., a mammal (e.g., a human or a non-human primate, a cow, horse, pig, sheep, cat, dog, mouse, or rat), a bird (e.g., chicken), or a fish. A biological sample can be any tissue or bodily fluid obtained from the biological organism, e.g., blood, a blood fraction, or a blood product (e.g., serum, plasma, platelets, red blood cells, and the like), sputum or saliva, tissue (e.g., kidney, lung, liver, heart, brain, nervous tissue, thyroid, eye, skeletal muscle, cartilage, or bone tissue); cultured cells, e.g., primary cultures, explants, and transformed cells, stem cells, or cells found in stool, urine, etc.

In some embodiments, DTME is applied to fix isolated nuclei, which can be from any type of cells, for example as described above. Methods of forming isolated nuclei are known and can be used as desired. Exemplary methods of generating isolated nuclei include those described in U.S. Pat. No. 8,546,134; Gaublomme, et al., Nature Communications volume 10, Article number: 2907 (2019).

In yet other embodiments, DTME is applied to fix isolated extracellular vesicles, which can be from any type of cells or biological samples, for example as described above. Methods for isolation of extracellular vesicles are described in, for example, Brennan et al., Sci Rep 10, 1039 (2020).

In some embodiments, the cells are also permeabilized to allow for diffusion or reagents (e.g., RT reagents) into the cells. Permeabilization can remove cellular membrane lipids to allow large molecules such as enzymes to enter the cell. In some embodiments, a detergent is used for permeabilization. Exemplary detergents can include, for example, Triton X-100 and NP-40 are used for permeabilization (for example, at 0.1-0.5% (v/v, in PBS). In some embodiments, a steroidal saponin (or saraponin) is used to solubilize lipid, resulting in permeabilization. An exemplary saraponin is Digitonin.

In some embodiments, fixed and permeabilized single cells, nuclei or extracellular vesicles are distributed into partitions. Methods and compositions for partitioning are described, for example, in published patent applications WO 2010/036352, US 2010/0173394, US 2011/0092373, and US 2011/0092376. The plurality of partitions can be, for example, a plurality of emulsion droplets, or a plurality of microwells, etc.

In some embodiments, one or more reagents are added during droplet formation or to the droplets after the droplets are formed. Methods and compositions for delivering reagents to one or more partitions include microfluidic methods as known in the art; droplet or microcapsule combining, coalescing, fusing, bursting, or degrading (e.g., as described in U.S. 2015/0027,892; US 2014/0227,684; WO 2012/149,042; and WO 2014/028,537); droplet injection methods (e.g., as described in WO 2010/151,776); and combinations thereof.

The partitions can be picowells, nanowells, or microwells. The partitions can be pico-, nano-, or micro-reaction chambers, such as pico, nano, or microcapsules. The partitions can be pico-, nano-, or micro-channels. The partitions can be gel beads in aqueous solution (e.g., without an oil phase). Gel beads can comprise, for example, agarose or other hydrogel materials. Examples of such gel beads are described in, e.g., Nishikawa et al., ISME Communications volume 2, Article number: 92 (2022) The partitions can be droplets, e.g., emulsion droplets. In some embodiments, a droplet comprises an emulsion composition, i.e., a mixture of immiscible fluids (e.g., water and oil). In some embodiments, a droplet is an aqueous droplet that is surrounded by an immiscible carrier fluid (e.g., oil). In some embodiments, a droplet is an oil droplet that is surrounded by an immiscible carrier fluid (e.g., an aqueous solution). In some embodiments, the droplets described herein are relatively stable and have minimal coalescence between two or more droplets. In some embodiments, less than 0.0001%, 0.0005%, 0.001%, 0.005%, 0.01%, 0.05%, 0.1%, 0.5%, 1%, 2%, 3%, 4%, 5%. 6%, 7%. 8%, 9%, or 10% of droplets generated from a sample coalesce with other droplets. The emulsions can also have limited flocculation, a process by which the dispersed phase comes out of suspension in flakes. In some cases, such stability or minimal coalescence is maintained for up to 4, 6, 8, 10, 12, 24, or 48 hours or more (e.g., at room temperature, or at about 0, 2, 4, 6, 8, 10, or 12° C.). In some embodiments, the droplet is formed by flowing an oil phase through an aqueous sample or reagents.

The oil phase of an emulsion can comprise a fluorinated base oil which can additionally be stabilized by combination with a fluorinated surfactant such as a perfluorinated polyether. Exemplary oil phase compositions along these lines are described in, e.g., PCT WO 2020/247950 and US Patent Publication No. 2017/0175179.

In some embodiments, the sample is partitioned into, or into at least, 500 partitions, 1000 partitions, 2000 partitions, 3000 partitions, 4000 partitions, 5000 partitions, 6000 partitions, 7000 partitions, 8000 partitions, 10,000 partitions, 15,000 partitions, 20,000 partitions, 30,000 partitions, 40,000 partitions, 50,000 partitions, 60,000 partitions, 70,000 partitions, 80,000 partitions, 90,000 partitions, 100,000 partitions, 200,000 partitions, 300,000 partitions, 400,000 partitions, 500,000 partitions, 600,000 partitions, 700,000 partitions, 800,000 partitions, 900,000 partitions, 1,000,000 partitions, 2,000,000 partitions, 3,000,000 partitions, 4,000,000 partitions, 5,000,000 partitions, 10,000,000 partitions, 20,000,000 partitions, 30,000,000 partitions, 40,000,000 partitions, 50,000,000 partitions, 60,000,000 partitions, 70,000,000 partitions, 80,000,000 partitions, 90,000,000 partitions, 100,000,000 partitions, 150,000,000 partitions, or 200,000,000 partitions.

In some embodiments, the droplets that are generated are substantially uniform in shape and/or size. For example, in some embodiments, the droplets are substantially uniform in average diameter. In some embodiments, the droplets that are generated have an average diameter of about 0.001 microns, about 0.005 microns, about 0.01 microns, about 0.05 microns, about 0.1 microns, about 0.5 microns, about 1 microns, about 5 microns, about 10 microns, about 20 microns, about 30 microns, about 40 microns, about 50 microns, about 60 microns, about 70 microns, about 80 microns, about 90 microns, about 100 microns, about 150 microns, about 200 microns, about 300 microns, about 400 microns, about 500 microns, about 600 microns, about 700 microns, about 800 microns, about 900 microns, or about 1000 microns. In some embodiments, the droplets that are generated have an average diameter of less than about 1000 microns, less than about 900 microns, less than about 800 microns, less than about 700 microns, less than about 600 microns, less than about 500 microns, less than about 400 microns, less than about 300 microns, less than about 200 microns, less than about 100 microns, less than about 50 microns, or less than about 25 microns. In some embodiments, the droplets that are generated are non-uniform in shape and/or size.

In some embodiments, the droplets that are generated are substantially uniform in volume. For example, the standard deviation of droplet volume can be less than about 1 picoliter, 5 picoliters, 10 picoliters, 100 picoliters, 1 nL, or less than about 10 nL. In some cases, the standard deviation of droplet volume can be less than about 10-25% of the average droplet volume. In some embodiments, the droplets that are generated have an average volume of about 0.001 nL, about 0.005 nL, about 0.01 nL, about 0.02 nL, about 0.03 nL, about 0.04 nL, about 0.05 nL, about 0.06 nL, about 0.07 nL, about 0.08 nL, about 0.09 nL, about 0.1 nL, about 0.2 nL, about 0.3 nL, about 0.4 nL, about 0.5 nL, about 0.6 nL, about 0.7 nL, about 0.8 nL, about 0.9 nL, about 1 nL, about 1.5 nL, about 2 nL, about 2.5 nL, about 3 nL, about 3.5 nL, about 4 nL, about 4.5 nL, about 5 nL, about 5.5 nL, about 6 nL, about 6.5 nL, about 7 nL, about 7.5 nL, about 8 nL, about 8.5 nL, about 9 nL, about 9.5 nL, about 10 nL, about 11 nL, about 12 nL, about 13 nL, about 14 nL, about 15 nL, about 16 nL, about 17 nL, about 18 nL, about 19 nL, about 20 nL, about 25 nL, about 30 nL, about 35 nL, about 40 nL, about 45 nL, or about 50 nL.

In other embodiments, partitions are not used in the methods and composition described herein. For example, instead of single cells in partitions, a biological sample, for example, which can be individual cells or a tissue, can be fixed with DTME. In some embodiments, the methods described herein can be used for spatial profiling. Spatial profiling is a method for highly multiplex spatial profiling of proteins or RNAs suitable for use on fixed, optionally paraffin-embedded samples. See, e.g., Beecham, Methods Mol Biol. 2055:563-583 (2020). In some embodiments, the methods described herein allow to improved spatial profiling methods by using in situ revers transcription in a DTME-fixed tissue sample. The resulting cDNAs can be detected and quantified. In some embodiments, the cDNAs and optionally also gDNA from the cells, can be barcoded, fragmented with a tagmentase and detected by sequencing.

In some embodiments, cell-specific barcodes can be linked to the cells themselves and used to barcode nucleic acids from the cells. For example, cell-specific barcodes can be synthesized on cells or nuclei for example using a split and pool method. For example, an oligonucleotide comprising a common sequence can be attached to cell or nuclear membranes or cells or nuclei, respectively, to form a mixture of cells or nuclei having the oligonucleotide in attached to the membranes. The mixture can then be split into portions, where each portion receives a different nucleotide added to the oligonucleotide. The cells or nuclei are then combined, mixed, and split into portions again. This process if repeated, resulting in a unique, cell-specific (or nuclei-specific) nucleotide sequence on the cells or nuclei. An example of split-and-pool methods is provided in Fan, et al., Science 2015 Feb. 6; 347(6222):1258367. Optionally a common capture sequence can be added to the 3′ end of the oligonucleotides, such that the resulting oligonucleotides include a 5′ common sequence (optionally usable as a PCR handle, a cell-specific barcode, and a 3′ capture sequence. These can be used, for example in an in situ RT reaction as described below.

Following fixation with DTME and permeabilization, and whether in partitions or not (e.g., as single cells or in a tissue), in situ reverse transcription can be performed on the fixed cells, nuclei or extracellular vesicles. In situ reverse transcription can be performed as desired to form cDNAs from mRNA, miRNA, hnRNA snoRNA, piRNA, or any other form of RNA as desired. A variety of reverse transcriptases and conditions can be used to perform in situ reverse transcription.

Reverse transcription (RT) is an amplification method that copies RNA into DNA. RT reactions can be performed with reaction mixtures as desired. For example, the disclosure provides for reverse transcribing one or more RNA (including for example, all RNA in a cell, e.g., to make a cDNA library, or targeted RNA sequences) under conditions to allow for reverse transcription and generation of a first strand cDNA. The RT reaction can be primed with an RT primer that primes an RT reaction from at least one target RNA molecule. Exemplary RT primers can include, but are not limited to, those having a 3′ end sequence that anneals to target RNA. For example the 3′ end sequence can be one that is random, an oligo dT (also referred to herein as a “poly T”) sequence, or an RNA-specific sequence. Oligo dT sequences are single stranded sequences of deoxythymine (dT). The length of the oligo dT sequence can vary, for example from 6 bases to 30 bases and may be a mixture of oligo dT sequences with different lengths. Components and conditions for RT reactions are generally known.

In addition to a 3′ end sequence that anneals to the target RNA, the RT primer can also include one or more barcode sequence (for example, at least a partition-specific barcode as described herein) and optionally a PCR handle sequence, i.e., a sequence that can be added to the end of all resulting cDNA sequences such that the entire set of different cDNAs can be amplified later with a universal primer. Exemplary PCR handle sequences can include but are not limited to universal sequences used in various sequencing platforms, including but not limited to the P5/P7 sequences from Illumina (e.g., P5 primer CGACGCTCTTCCGATCT (SEQ ID NO: 6) and P7 primer CGTGTGCTCTTCCGATCT (SEQ ID NO: 7)), PacificBio, or Ion Torrent. The barcode sequences can include, for example, a sample, partition-specific (e.g., bead-specific for embodiments in which individual beads carrying oligonucleotides are introduced into partitions) and/or sample barcode.

RT amplification reaction mixtures can be prepared as is known. In some embodiments, the amplification reaction mixture comprises one or more target-specific amplification primers. In some embodiments, the amplification mixture further comprises one or more of salts, nucleotides, buffers, stabilizers, reverse transcriptase, DNA polymerase, a detectable agent, and nuclease-free water. Exemplary methods of digital RT-PCR are described in, e.g., Sedlak et al., J Clin Microbiol 55:442-449 (2014).

Suitable reverse transcriptases can include but are not limited to Maxima RNAse+ (Thermo), Maxima RNAse− (Thermo), murine leukemia virus (MLV) reverse transcriptase (Gerard and Grandgenett, Journal of Virology 15:785-797, 1975; Verma, Journal of Virology 15:843-854, 1975) or SEQ ID NO:1, feline leukemia virus (FLV) reverse transcriptase (Rho and Gallo, Cancer Lett., 10:207-221, 1980 or SEQ ID NO: 1, bovine leukemia virus (BLV) (Demirhan et al., Anticancer Res., 16:2501-5, 1996; Drescher et al., Arch Geschwulstforsch., 49:569-79, 1979), Avian Myeloblastosis Virus (AMV) reverse transcriptase, Respiratory Syncytial Virus (RSV) reverse transcriptase, Equine Infectious Anemia Virus (EIAV) reverse transcriptase, Rous-associated Virus-2 (RAV2) reverse transcriptase, SUPERSCRIPT II reverse transcriptase, SUPERSCRIPT III reverse transcriptase (U.S. Pat. Nos. 8,541,219, 7,056,716, 7,078,208), THERMOSCRIPT reverse transcriptase and MMLV RNase H-reverse transcriptase and Sensiscript (Qiagen).

In some embodiments, a second cDNA strand is formed following first strand cDNA synthesis. For example, a polymerase can extend a primer that anneals to a region (for example a 3′ region) of the first strand cDNA to form a second strand cDNA. In some embodiments, if the primer used to form the first strand cDNA did not include a partition-specific barcode, such a barcode can be included in the primer used to initiate formation of the second strand cDNA. The first or second or both strands of the cDNA can be amplified and if desired amplification can result in universal sequences and optionally barcode sequences being added to one or both ends of the cDNA. This can be used for example in preparation for next-generation sequencing.

In some embodiments, amplification such as PCR amplification can be used to amplify the cDNA. In some embodiments, the amplification reaction can be monitored in real-time, for example to quantify target RNA sequences in a cell or tissue. Real-time monitoring can include generation of a fluorescent signal during amplification and monitoring a change in fluorescent signal as a function of cycles of amplification. Examples of real-time PCR can include, for example inclusion of Tagman-based probes or for example molecular beacons.

In some embodiments, RNA/first strand cDNA duplexes, or first strand cDNA/second strand cDNA duplexes can be fragmented using a transposase that links oligonucleotides to the ends of the resulting fragments, allowing for attachment of common sequences on the fragments that can be used for, for example, manipulation and amplification of the fragments, e.g., for sequencing. Thus in some embodiments, the methods can include contacting the RNA/DNA hybrids with contacting a transposase that introduces heterologous end adaptor oligonucleotides into the RNA/DNA hybrids to form DNA fragments, and optionally RNA fragments, comprising the heterologous end adaptor oligonucleotides; or contacting the double-stranded cDNA with contacting a transposase that introduces heterologous end adaptor oligonucleotides into the double-stranded DNA to form cDNA fragments comprising the heterologous end adaptor oligonucleotides.

Fragmentation and attachment of end adaptors on the RNA/first strand cDNA hybrid molecules or DNA/cDNA hybrid molecules can be achieved with a transposase. The action of the transposase sometimes referred to as “tagmentation” and can involve introduction of different adapter sequences on different sides of a DNA breakage point caused by the transposase or the adapter sequences added can be identical. In either case, the one or two adapter sequences are common adapter sequences in that the adapter sequences are the same across a diversity of DNA fragments. Homoadapter-loaded tagmentases are tagmentases that contain adapters of only one sequence, which adapter is added to both ends of a tagmentase-induced breakpoint in the genomic DNA. Heteroadapter-loaded tagmentases are tagmentases that contain two different adapters, such that a different adapter sequence is added to the two DNA ends created by a tagmentase-induced breakpoint in the DNA. Adapter loaded tagmentases are further described, e.g., in U.S. Patent Publication Nos: 2010/0120098; 2012/0301925; and 2015/0291942 and U.S. Pat. Nos: 5,965,443; U.S. Pat. Nos. 6,437,109; 7,083,980; 9,005,935; and 9,238,671, the contents of each of which are hereby incorporated by reference in the entirety for all purposes. Tagmentation of RNA/DNA hybrids is described in, e.g., Bo LuLiting et al., eLife 9:e54919 (2020).

A tagmentase is an enzyme that is capable of forming a functional complex with a transposon end-containing composition and catalyzing insertion or transposition of the transposon end-containing composition into the double-stranded target DNA with which it is incubated in an in vitro transposition reaction. Exemplary transposases include but are not limited to modified Tn5 transposases that are hyperactive compared to wildtype Tn5, for example can have one or more mutations selected from E54K, M56A, or L372P. Wild-type Tn5 transposon is a composite transposon in which two near-identical insertion sequences (IS50L and IS50R) are flanking three antibiotic resistance genes (Reznikoff W S. Annu Rev Genet 42: 269-286 (2008)). Each IS50 contains two inverted 19-bp end sequences (ESs), an outside end (OE) and an inside end (IE). However, wild-type ESs have a relatively low activity and were replaced in vitro by hyperactive mosaic end (ME) sequences. A complex of the transposase with the 19-bp ME is thus all that is necessary for transposition to occur, provided that the intervening DNA is long enough to bring two of these sequences close together to form an active Tn5 transposase homodimer (Reznikoff W S., et al. Mol Microbiol 47: 1199-1206 (2003)). Transposition is a very infrequent event in vivo, and hyperactive mutants were historically derived by introducing three missense mutations in the 476 residues of the Tn5 protein (E54K, M56A, L372P), which is encoded by IS50R (Goryshin I Y, Reznikoff W S. 1998. J Biol Chem 273: 7367-7374 (1998)). Transposition works through a “cut-and-paste” mechanism, where the Tn5 excises itself from the donor DNA and inserts into a target sequence, creating a 9-bp duplication of the target (Schaller H. Cold Spring Harb Symp Quant Biol 43: 401-408 (1979); Reznikoff W S., Annu Rev Genet 42: 269-286 (2008)). In current commercial solutions (Nextera™ DNA kits, Illumina), free synthetic ME adapters are end-joined to the 5′-end of the target DNA by the transposase (tagmentase).

In some embodiments, the adapter(s) is at least 19 nucleotides in length, e.g., 19-100 nucleotides. In some embodiments, the adapters are double stranded with a 5′ end overhang, wherein the 5′ overhand sequence is different between heteroadapters, while the double stranded portion (typically 19 bp) is the same. In some embodiments, an adapter comprises TCGTCGGCAGCGTC (SEQ ID NO: 1) or GTCTCGTGGGCTCGG (SEQ ID NO:2). In some embodiments involving the heteroadapter-loaded tagmentase, the tagmentase is loaded with a first adapter comprising TCGTCGGCAGCGTC (SEQ ID NO:1) and a second adapter comprising GTCTCGTGGGCTCGG (SEQ ID NO:2). In some embodiments, the adapter comprises AGATGTGTATAAGAGACAG (SEQ ID NO:3) and the complement thereof (this is the mosaic end and this is the only specifically required cis active sequence for Tn5 transposition). In some embodiments, the adapter comprises TCGTCGGCAGCGTCAGATGTGTATAAGAGACAG (SEQ ID NO:4) with the complement for AGATGTGTATAAGAGACAG (SEQ ID NO:3) or GTCTCGTGGGCTCGGAGATGTGTATAAGAGACAG (SEQ ID NO:5) with the complement for AGATGTGTATAAGAGACAG (SEQ ID NO:3). In some embodiments involving the heteroadapter-loaded tagmentase, the tagmentase is loaded with a first adapter comprising TCGTCGGCAGCGTCAGATGTGTATAAGAGACAG (SEQ ID NO:4) with the complement for AGATGTGTATAAGAGACAG (SEQ ID NO:3) and GTCTCGTGGGCTCGGAGATGTGTATAAGAGACAG (SEQ ID NO: 5) with the complement for AGATGTGTATAAGAGACAG (SEQ ID NO: 3).

In some embodiments, the transposase generates random breaks in the hybrid molecules and randomly inserts at the breaks first adaptor oligonucleotides or second adaptor oligonucleotides, thereby forming hybrid molecule fragments comprising (i) a first 5′ end linked to a first adaptor oligonucleotide and (ii) a first 3′ end and (iii) a second 5′ end linked to a second adaptor oligonucleotide and (iv) a second 3′ end, wherein the first adaptor oligonucleotide comprises a first universal sequence and the second adaptor oligonucleotide comprises a second universal sequence. In some embodiments, the transposase is used loaded as a heteroadaptor such that two different adaptors can be attached to breaks in the target hybrid nucleic acid. Alternatively, two transposases can be used wherein a first transposase is loaded with one set of adaptor oligonucleotides (e.g., for ease of description the “first adaptor oligonucleotide”) and a second transposase is loaded with a second set of adaptor oligonucleotides, (the “second adaptor oligonucleotide”) thereby allowing, by random fragmentation by the two transposases, the formation of fragments that have the first adaptor oligonucleotide on a first end and the second adaptor on the second end. An “adaptor oligonucleotide” refers to an oligonucleotide that carries a universal sequences, wherein the universal sequences are common to end sequences between different fragments to which the oligonucleotide adaptor is attached and permit their use as PCR handle sequences, allowing one pair of universal primers to amplify different fragments that have the universal sequences.

Fragmentation by tagmentation can occur within the fixed and permeabilized cells, nuclei or vesicles, for example, prior to introduction of the cells, nuclei or vesicles into partitions. If it is desired to also sequence genomic DNA from the cells or nuclei, conditions of tagmentation can be selected such that tagmentation of the gDNA in the cells or nuclei occurs to generate gDNA fragments having adapter oligonucleotides at their ends, for example as described above in the context of cDNA hybrid molecules. This can allow for both gDNA and cDNA fragments comprising the same adapter oligonucleotides, and allowing for subsequent cell-specific barcoding using the oligonucleotides comprising partition-specific barcodes to be linked to the adapter oligonucleotides on both gDNA and cDNA fragments, which can subsequently be processed together for sequencing, allowing for generation of both RNA and gDNA sequencing data.

In some embodiments, once amplicons comprising the adapter-end labelled DNA fragments are formed, the amplicons can be combined, for example contents of the partitions can be combined, to form a bulk solution comprising the contents of a plurality of the cells, nuclei or vesicles. The amplicons in the resulting bulk solution can be nucleotide sequenced. Methods for high throughput sequencing and genotyping are known in the art. For example, such sequencing technologies include, but are not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety.

Exemplary DNA sequencing techniques include fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the present technology provides parallel sequencing of partitioned amplicons (PCT Publication No. WO 2006/0841,32, herein incorporated by reference in its entirety). In some embodiments, DNA sequencing is achieved by parallel oligonucleotide extension (See, e.g., U.S. Pat. Nos. 5,750,341; and 6,306,597, both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; and U.S. Pat. Nos. 6,432,360; 6,485,944; 6,511,803; herein incorporated by reference in their entireties), the 454 picotiter pyrosequencing technology (Margulies et al., 2005 Nature 437, 376-380; U.S. Publication No. 2005/0130173; herein incorporated by reference in their entireties), the Solexa single base addition technology (Bennett et al., 2005, Pharmacogenomics, 6, 373-382; U.S. Pat. Nos. 6,787,308; and 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 2000/018957; herein incorporated by reference in its entirety).

Typically, high throughput sequencing methods share the common feature of massively parallel, high-throughput strategies, with the goal of lower costs in comparison to older sequencing methods (See, e.g., Voelkerding et al., Clinical Chem., 55: 641-658, 2009; MacLean et al., Nature Rev. Microbiol., 7:287-296; each herein incorporated by reference in their entirety). Such methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbial., 7:287-296; U.S. Pat. Nos. 6,210,891; and 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55. 641-658, 2009; MacLean et al., Nature Rev. Microbial., 7:287-296; U.S. Pat. Nos. 6,833,246; 7,115,400; and 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbial., 7:287-296; U.S. Pat. Nos. 5,912,148; and 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing is employed (See, e.g., Astier et al., J. Am. Chem. Soc. 2006 Feb. 8; 128(5)1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, HeliScope by Helicos BioSciences is employed (Voelkerding et al., Clinical Chem., 55. 641-658, 2009; MacLean et al., Nature Rev. Microbial, 7:287-296; U.S. Pat. Nos. 7,169,560; 7,282,337; 7,482,120; 7,501,245; 6,818,395; 6,911,345; and 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (See, e.g., Science 327(5970): 1190 (2010); U.S. Pat. Appl. Pub. Nos. 2009/0026082; 2009/0127589; 2010/0301398; 2010/0197507; 2010/0188073; and 2010/0137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers the hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per base accuracy of the Ion Torrent sequencer is ˜99.6% for 50 base reads, with ˜100 Mb generated per run. The read-length is 100 base pairs. The accuracy for homopolymer repeats of 5 repeats in length is ˜98%. The benefits of ion semiconductor sequencing are rapid sequencing speed and low upfront and operating costs.

Another exemplary nucleic acid sequencing approach that may be adapted for use with the present invention was developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 2009/0035777, which is incorporated herein in its entirety.

Other single molecule sequencing methods include real-time sequencing by synthesis using a VisiGen platform (Voelkerding et al., Clinical Chem., 55: 641-58, 2009; U.S. Pat. No. 7,329,492; and U.S. patent application Ser. Nos. 11/671,956; and 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

Another real-time single molecule sequencing system developed by Pacific Biosciences (Voelkerding et al., Clinical Chem., 55. 641-658, 2009; MacLean et al., Nature Rev. Microbiol., 7:287-296; U.S. Pat. Nos. 7,170,050; 7,302,146; 7,313,308; and 7,476,503; all of which are herein incorporated by reference) utilizes reaction wells 50-100 nm in diameter and encompassing a reaction volume of approximately 20 zeptoliters (10⁻²¹ L). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

In certain embodiments, the single molecule real time (SMRT) DNA sequencing methods using zero-mode waveguides (ZMWs) developed by Pacific Biosciences, or similar methods, are employed. With this technology, DNA sequencing is performed on SMRT chips, each containing thousands of zero-mode waveguides (ZMWs). A ZMW is a hole, tens of nanometers in diameter, fabricated in a 100 nm metal film deposited on a silicon dioxide substrate. Each ZMW becomes a nanophotonic visualization chamber providing a detection volume of just 20 zeptoliters (10⁻²¹ L). At this volume, the activity of a single molecule can be detected amongst a background of thousands of labeled nucleotides. The ZMW provides a window for watching DNA polymerase as it performs sequencing by synthesis. Within each chamber, a single DNA polymerase molecule is attached to the bottom surface such that it permanently resides within the detection volume. Phospholinked nucleotides, each type labeled with a different colored fluorophore, are then introduced into the reaction solution at high concentrations which promote enzyme speed, accuracy, and processivity. Due to the small size of the ZMW, even at these high concentrations, the detection volume is occupied by nucleotides only a small fraction of the time. In addition, visits to the detection volume are fast, lasting only a few microseconds, due to the very small distance that diffusion has to carry the nucleotides. The result is a very low background.

Processes and systems for such real time sequencing that may be adapted for use with the invention are described in, for example, U.S. Pat. Nos. 7,405,281; 7,315,019; 7,313,308; 7,302,146; and 7,170,050; and U.S. Pat. Pub. Nos. 2008/0212960; 2008/0206764; 2008/0199932; 2008/0199874; 2008/0176769; 2008/0176316; 2008/0176241; 2008/0165346; 2008/0160531; 2008/0157005; 2008/0153100; 2008/0153095; 2008/0152281; 2008/0152280; 2008/0145278; 2008/0128627; 2008/0108082; 2008/0095488; 2008/0080059; 2008/0050747; 2008/0032301; 2008/0030628; 2008/0009007; 2007/0238679; 2007/0231804; 2007/0206187; 2007/0196846; 2007/0188750; 2007/0161017; 2007/0141598; 2007/0134128; 2007/0128133; 2007/0077564; 2007/0072196; and 2007/0036511; and Korlach et al. (2008) “Selective aluminum passivation for targeted immobilization of single DNA polymerase molecules in zero-mode waveguide nanostructures” PNAS 105(4): 1176-81, all of which are herein incorporated by reference in their entireties.

Also provided are cells, nuclei and extracellular vesicles fixed with DTME as described herein. In some embodiments, the fixed and permeabilized cells, nuclei or extracellular vesicles comprise a heterologous reverse transcriptase and optionally reagents for reverse transcription, e.g., an oligonucleotide primer (e.g., as described herein), dNTPs and buffers for supporting reverse transcription reactions.

### Example

General activity and efficiency of in situ reverse transcription (RT) reaction of DSP and DTME fixed cells was determined by using real-time quantitative PCR (qPCR) of intron-spanning amplicon assays of 12 reference genes that were identified in 20 widely used human cell lines including HeLa, MCF-7, A-549, K-562, HL-60(TB), HT-29, MDA-MB-231, HCT 116, U-937, SH-SY5Y, U-251MG, MOLT-4, RPMI-8226, HEK293, MRC-5, HUVEC/TERT2, HMEC, HFF-1, HUES 9, XCL-1 (Ricz et al., Scientific Reports (2021) 11:19459).

In brief, cells were washed with PBS supplemented with RNase Inhibitor (PBSRI) and then divided into two portions, Test and Control. In the Test, cells were resuspended with either DSP or DTME fixative of final concentration 600 ng fixative/1000 cells/l in PBS solution at room temperature for 30 min. Fixation was stopped with the supplement of amine-based quenchers for 30 min incubation on ice. Cells were then permeabilized with common detergents, such as Tween20 and Digitonin. Real-time quantitative PCR of 12 reference genes amplicon assays were conducted by using cDNA samples derived from the fixed-cell in-situ reverse transcription reaction (Test Sample) and the commercial iScript Advanced cDNA Synthesis Kit (Control Sample), respectively. The qPCR assays were performed in duplicate with four 10-fold serial diluted inputs to establish a titration curve for amplification efficiency determination. The level of threshold cutoff was determined by the algorithm and fixed among all samples for the Cq value determination and comparison. Data of at least three independent experimental repeats were shown in Table 1.

Table 1 shows the AACq value of 12 qPCR assays of targets: SNW1, CNOT4, PUM1, PCBP1, IP08, HNRNPL, TBP, UBC, PPIA, RPL30, ACTB, and GAPDH. The real-time quantitative PCR results were generated by using cDNA samples derived from the in situ reverse transcription reaction of either DSP or DTME fixed cells (Test Sample), and cDNA samples derived from the commercial reagent kit of cells in bulk (Control Sample). Reverse transcription of Test and Control samples was further conducted in the presence and absence of reverse transcriptase supplement to make sure the amplification is cDNA specific (i.e., Test with/without RTase, and Control with/without RTase). The amount of cDNA input of Test and Control was normalized per cell-equivalent base. The level of threshold cutoff was determined by algorithm and fixed among all samples for Cq value determination. The value of AACq was calculated based on the highest cDNA input concentration of the titration curve. The value shown in the Table 1 is an average AACq value of three independent experimental repeats. The first ACq value was calculated based on the Cq value difference of cDNA sample derived from reactions with and without Rtase supplement. The AACq value was calculated by subtracting the first ACq value of Test Sample with that of the Control Sample. Negative AACq value indicates earlier Cq value, thus higher yield of cDNA synthesis of the Test Sample as compared to that of the Control Sample. In contrast, positive AACq value indicates poor cDNA yield of Test versus Control. Value of minus 1 means 100% more of targeted cDNA copies of Test Sample as compared to that of the Control Sample. As shown in Table 1, using 12 universal reference genes as demonstration, the yield of cDNA synthesis of in-situ RT reaction using reversible-crosslinker DTME fixed cells was significantly higher than that of reversible-crosslinker DSP fixed cells.

TABLE 1

| ΔΔCq Value | DSP-fixed cells | DTME-fixed cells |
|---|---|---|
| of Assay | (Test minus Control) | (Test minus Control) |
| SNW1 | −0.5 | −1.6 |
| CNOT4 | −0.3 | −3.0 |
| PUM1 | −1.5 | −3.9 |
| PCBP1 | 1.3 | −1.1 |
| IPO8 | 1.5 | −2.0 |
| HNRNPL | 2.0 | −2.1 |
| TBP | 0.2 | −1.7 |
| UBC | 1.5 | −1.0 |
| PPIA | −0.1 | −0.9 |
| RPL30 | 2.4 | 1.0 |
| ACTB | −4.9 | −12.1 |
| GAPDH | 0.3 | −2.0 |

Although the foregoing disclosure has been described in some detail by way of illustration and example for purposes of clarity of understanding, one of skill in the art will appreciate that certain changes and modifications may be practiced within the scope of the appended claims. In addition, each reference provided herein, including patents, patent applications, non-patent literature, and Genbank accession numbers, is incorporated by reference in its entirety to the same extent as if each reference was individually incorporated by reference. Where a conflict exists between the instant application and a reference provided herein, the instant application shall dominate.

What is claimed is:

1. A method of in situ reverse transcription, the method comprising,
contacting a single cell or a plurality of cells or nuclei or extracellular vesicles with dithiobismaleimidoethane (DTME) in an amount sufficient to fix the cells;
permeabilizing the cells or nuclei or extracellular vesicles;
introducing a reverse transcriptase into the cells or nuclei or extracellular vesicles to form first strand cDNAs from the RNAs in the cells or nuclei or extracellular vesicles, thereby conducting in situ reverse transcription.

2. The method of claim 1, wherein the RNAs are mRNAs.

3. The method of claim 1, wherein the plurality of cells is in a plant tissue or animal tissue.

4. The method of claim 1, wherein the cells are individual cells and the individual cells are within separate partitions.

5. The method of claim 4, wherein the partitions are droplets, tubes, gel beads or microwells.

6. The method of claim 4, comprising linking partition-specific barcode oligonucleotides to the cDNAs or fragments or complements thereof.

7. The method of claim 1, further comprising amplifying the cDNAs or fragments thereof with a polymerase.

8. The method of claim 7, wherein the amplifying comprises polymerase chain reaction (PCR).

9. The method of claim 8, wherein the PCR is monitored in real-time.

10. The method of claim 1, wherein the RNAs and first strand cDNAs form RNA/DNA hybrids and the method further comprising contacting the RNA/DNA hybrids with contacting a transposase that introduces heterologous end adaptor oligonucleotides into the RNA/DNA hybrids to form DNA fragments, and optionally RNA fragments, comprising the heterologous end adaptor oligonucleotides.

11. The method of claim 1, further comprising the forming second strand cDNAs from the first strand cDNAs wherein the first strand cDNAs and second strand cDNAs form double-stranded DNA and the method further comprising contacting the double-stranded DNA with contacting a transposase that introduces heterologous end adaptor oligonucleotides into the double-stranded DNA to form DNA fragments comprising the heterologous end adaptor oligonucleotides.

12. The method of claim 1, wherein the cells are mammalian cells.

13. The method of claim 1, comprising storing the cells after the fixing and before the permeabilizing or the introducing.

14. The method of claim 1, wherein the RNAs and first strand cDNAs form RNA/DNA hybrids and the method further comprises:
introducing into the cells and nuclei of the cells transposases, wherein the transposases:
(i) introduce heterologous end adaptor oligonucleotides into genomic DNA in the nuclei to form gDNA fragments comprising the heterologous end adaptor oligonucleotides; and
(ii) introduces heterologous end adaptor oligonucleotides into the RNA/DNA hybrids to form cDNA fragments comprising the heterologous end adaptor oligonucleotides;
introducing the cells into partitions;
in the partitions, attaching partition-specific barcode oligonucleotides to the gDNA fragments comprising the heterologous end adaptor oligonucleotides and the cDNA fragments comprising the heterologous end adaptor oligonucleotides.

15. A plurality of fixed cells, wherein the cells were fixed with dithiobismaleimidoethane (DTME) and the fixed cells comprise a heterologous reverse transcriptase capable of generating cDNAs from mRNAs in the fixed cells.

16. The plurality of cells of claim 12, wherein the cells are mammalian cells.

17. The plurality of cells of claim 15, wherein individual cells of the plurality are contained in separate partitions.

18. The plurality of cells of claim 17, wherein the partitions are droplets or microwells.
